(19) United States
(12) Patent Application Publication
(10) Pub. No.: US 2013/0204333 A1
(43) Pub. Date: Aug. 8, 2013

(54) POWDERED MIX FOR USE IN THERAPY PACKS

(71) Applicant: Lloyd Huff, Dayton, OH (US)

(72) Inventor: Lloyd Huff, Dayton, OH (US)

(21) Appl. No.: 13/366,463

(22) Filed: Feb. 6, 2012

(51) Int. Cl. A61F 7/10; C09K 3/00

(52) U.S. Cl. 607/114; 252/70

(57) ABSTRACT

A powdered mix for use in a cold pack is provided which includes a phase change material, precipitated silica, and a flowability enhancer comprising a carbonate mineral. The powdered mix, when placed into a cold pack, exhibits a longer hold time than conventional cold packs.

### BACKGROUND OF THE INVENTION

Embodiments of the invention relate to a powdered mix for use in a therapy pack, and more particularly, to a therapy pack including a phase change material, silica, and a third component which maintains the flexibility of the pack upon freezing.

Therapy packs such as cold packs are known for use in a variety of therapeutic applications including the treatment of sprains and strains, pulled muscles, sports injuries, headaches, post-operative pain, and pain from dental procedures. For example, the application of a cold pack applied immediately following an injury such as a sprained ankle is very effective in reducing swelling and inflammation. Commonly used cold packs typically include a water-based or organic liquid-based gel. Cold gel products are typically charged by placing the products in a freezer, and often include a freezing point depressant so that they remain soft and pliable when charged and ready for use.

Another composition which provides cold therapy and remains relatively conformable below the freezing point of water is a pack containing water as the phase change material in combination with a silica powder. See, for example, Salyer, U.S. Pat. Nos. 5,211,949 and 5,282,994. This composition uses the latent heat of fusion/crystallization of the water/ice in the powder, and has a longer hold time (i.e., maintains a desired temperature range) compared with gel packs. However, in order to achieve a long hold time, the water/silica powder must contain a relatively high concentration of water (about 60 to 75% by weight). At such concentrations, a cold pack comprising such a water/silica powder will be relatively stiff when first removed from the freezer. While the flexibility of a water/silica cold pack can be improved by reducing the water concentration, this consequently reduces the hold time of the cold pack.

The flexibility of the water/silica cold pack can also be improved by adding a freezing point depressant such as sodium chloride, but this significantly reduces the latent heat capacity of the water and also reduces the hold time.

Other known cold pack products use clay as the water containment medium. Such packs are very pliable after removal from the freezer, but the absorptive capacity of the clay material is limited, and this product has a relatively short hold time compared to a water/silica cold pack. The hold time may be increased by increasing the water concentration, but this results in reduced flexibility of the pack upon removal from a freezer.

Another problem with water/silica cold packs is that some silica powders have the tendency to absorb a considerable amount of air when mixed with water. The air is released over time in the freeze/thaw process and can cause the sealed cold pack to balloon during use, making the pack difficult to apply/conform properly around the body part to which it is applied. This problem can be avoided or at least partially mitigated by vibrating the powder during the filling process, but this step adds to manufacturing costs.

Accordingly, there is a need in the art for a water/silica therapy pack for use in cold applications having flexibility upon removal from a freezer, which exhibits longer hold times as compared with existing gel packs, and which does not balloon over time.

### SUMMARY OF THE INVENTION

Embodiments of the invention meet that need by providing a powdered mix for use in a cold pack which includes a phase change material, precipitated silica and a third component comprising a natural mined carbonate material such as dolomite or calcium carbonate, which renders the powder more flowable in its frozen state. Cold packs including the mix have improved flexibility upon removal from a freezer, and do not suffer from the problem of ballooning due to the release of air. The cold pack also has a longer hold time when compared with conventional gel packs. By “hold time,” it is meant the amount of time the cold pack stays in a therapeutic temperature range.

According to one aspect of the invention, a powdered mix for use in a therapy pack is provided comprising a phase change material, precipitated silica particles, and a flowability enhancer comprising a carbonate mineral. Preferably, the carbonate mineral is selected from the group consisting of dolomite and calcium carbonate.

Preferably, the powdered mix comprises from about 50 to 70% by weight of the phase change material, from about 25 to 40% by weight of the silica particles, and from about 10 to 20% by weight of the flowability enhancer.

In one embodiment, the phase change material comprises water.

The precipitated silica particles in the powdered mix preferably have a surface area of from about 140 to about 190 m²/g and an average particle size of about 250 microns.

In another embodiment of the invention, a cold pack is provided comprising a a sealed outer flexible polymeric film containing a powdered mix of a phase change material, a precipitated silica, and a flowability enhancer comprising a carbonate mineral. Preferably, the outer polymeric film is selected from the group consisting of polyesters, butadiene acrylonitrile copolymers, vinyl polymers, polyethylene, and ethylene-vinyl acetate copolymers.

Accordingly, it is a feature of embodiments of the invention to provide a powdered mix for use in a cold pack which exhibits good flexibility upon removal from a freezer, and to a cold pack containing the powdered mix.

Other features and advantages of the invention will be apparent from the following description, the accompanying drawings, and the appended claims.

### BRIEF DESCRIPTION OF THE DRAWINGS

FIG. 1 is graph illustrating the hold time for conventional cold packs in comparison with a cold pack prepared according to an embodiment of the invention.

### DETAILED DESCRIPTION OF THE PREFERRED EMBODIMENTS

The addition of a flowability enhancer to a silica/water cold pack has been found to make the pack more flexible upon removal from a freezer. In addition, it has been found that the use of a particular type of precipitated silica in combination with the flowability enhancer also contributes to flexibility in the frozen state as well as a longer hold time.

A preferred silica for use in the powdered mix is commercially available from PPG Industries, Inc. under the designation Hi-Sil® SC 72. This particular silica can absorb a large amount of liquid (up to 75%) and still remain in a flowable, dry powder state. The silica is an amorphous precipitated silica having a surface area of about 165 m²/g, an average particle size of about 200 to 300 microns, and preferably, about 250 microns, and a bulk density of about 185 g/l.

The Hi-Sil® SC 72 powder provides several advantages over the use of other precipitated silicas such as Hi-Sil® ABS, which have previously been used in cold packs. The SC 72 powder has a coarser, less fluffy structure than the ABS silica, produces less dusting upon agitation, and has a substantially larger particle size (250 μm compared with 35 μm for Hi-Sil® ABS). It is believed that the larger particle size of the silica prevents the powder from absorbing significant amounts of air during the mixing process, which can lead to ballooning once the mix is placed in a fluid impervious enclosure to form a cold pack.

The silica is preferably used in combination with a water phase change material. The freezing/melting point of water/ice at 0° C. is in the proper range for cold therapy, and water has a higher latent heat capacity (80 calories/g) than most other phase change materials. However, it should be appreciated that other phase change materials with freezing/melting points which are higher or lower than 0° C. may be used. Other suitable phase change materials include hydrocarbons, alcohols, glycerine, ethylene glycol, polyethylene glycol, clathrates, semi-clathrates, gas clathrates, hydrated salts, and combinations of these materials.

The powdered mix contains a third component which enhances flowability of the mix upon freezing. This flowability enhancer also aids in the prevention of swelling or ballooning when the powdered mix is contained in a cold pack. Preferred flowability enhancers are carbonate minerals such as dolomite and calcium carbonate which are relatively inexpensive, but it should be appreciated that any natural mined mineral materials may be used as long as they are not soluble in water, the powdered form does not absorb or adsorb a significant amount of the phase change material, and they provide the desired flowability to the powdered mix when placed in a cold pack and frozen.

While not wishing to be bound by theory, it is believed that the flowablility enhancer breaks up the crystalline structure of the frozen powder, rendering the powder more flowable in its frozen state.

The silica, phase change material, and flowability enhancer are preferably mixed together using a conventional commercial mixer. Preferably, the powdered mix comprises from about 50 to 70% by weight of the phase change material, from about 25 to 40% by weight of the silica particles, and from about 10 to 20% by weight of dolomite or calcium carbonate.

While embodiments of the invention described herein are directed to a powdered mix for use in a cold pack, it should be understood that the mix may also be used as a hot pack by heating the pack (preferably in a microwave oven). Where the phase change material is a non-polar material that does not absorb microwave energy, a polar material such as glycerin may be added to the powder composition to facilitate microwave heating as described in Salyer, U.S. Pat. No. 5,282,994, which is incorporated herein by reference. Ancillary devices may be used in conjunction with the mix to enhance performance, such as the use of an outer insulating layer to reduce heat flow or a strap or sleeve to hold the therapy pack in place.

It should also be understood that embodiments of the invention may be used for other heating and cooling applications such as inserts for cooling/warming vests, articles for soil and plant warming, warming blankets, and clothing articles.

In order that the invention may be more readily understood, reference is made to the following example, which is intended to be illustrative of the invention, but is not intended to be limiting in scope.

### EXAMPLE

A powdered cold pack was prepared in accordance with an embodiment of the invention using the following formulation:

| Component | Percent by Weight |
|---|---|
| Water | 54% |
| Hi-Sil ® SC-72 silica powder | 29% |
| Calcium carbonate powder | 17% |
| (325 mesh) | |

The water and silica powder were first mixed in a blade blender using 65 wt % water and 35 weight % silica. The calcium carbonate powder was then added and mixed such that the calcium carbonate comprised 17% by weight of the total mixture. The mixture was then placed in a 6.5 in.×11 in. plastic bag and sealed. The powder fill weight was 454 grams.

A water/ABS silica cold pack of the same dimensions was also prepared having a water concentration of 65% and a powder fill weight of 370 grams. The fill weight was chosen so that the water content of the water/SC-72/CaCO₃ pack was the same as the water/ABS silica cold pack.

A third cold pack was prepared to test the effect of a freezing point depressant on the water. The cold pack was identical to the water/ABS pack except that the water contained 6% by weight salt (sodium chloride). The fill weight of the salt water/ABS pack was 370 grams so that the water and salt water content of the packs were the same.

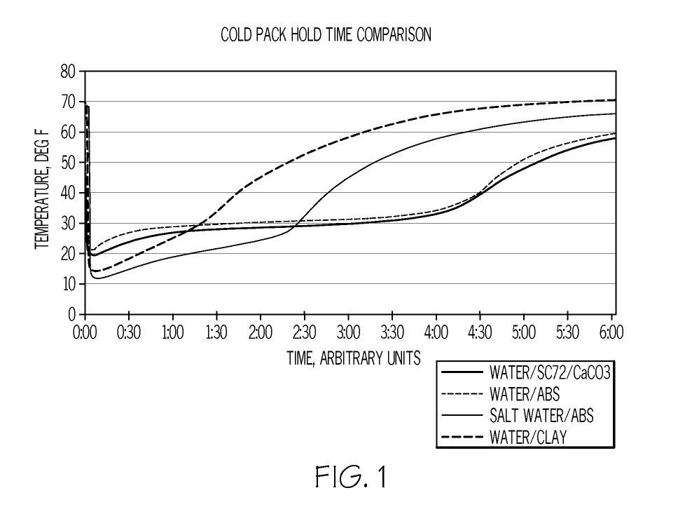

All three packs were placed in a freezer along with a commercially available water/clay powdered cold pack for about 12 hours and then removed. The out-of-freezer flexibility of the water/ABS silica pack was poor, with the pack being relatively stiff. In contrast, the commercial water/clay pack, the salt water/ABS pack, and water/SC-72/CaCO₃ pack were soft and conformable with excellent out-of-freezer flexibility. A bench top hold time test was then performed to compare the hold time of all four packs. The results of the test are shown in FIG. 1. As shown, the relative hold times of the water/ABS silica pack and the water/SC-72/CaCO₃ pack are essentially the same and significantly longer than that of the commercial water/clay pack and the salt water/ABS silica pack. Also as shown in FIG. 1, the water/SC-72/CaCO₃ cold pack provides cold therapy at nearly constant temperature for an extended period of time while possessing excellent out-of-freezer flexibility.

Having described the invention in detail and by reference to preferred embodiments thereof, it will be apparent that modifications and variations are possible without departing from the scope of the invention.

What is claimed is:

1. A powdered mix for use in a therapy pack comprising:
a phase change material;
precipitated silica particles; and
a flowability enhancer selected comprising a carbonate mineral.

2. The powdered mix of claim 1 wherein said carbonate mineral is selected from the group consisting of dolomite and calcium carbonate.

3. The powdered mix of claim 1 comprising from about 50 to 70% by weight of said phase change material.

4. The powdered mix of claim 1 comprising from about 25 to 40% by weight of said silica particles.

5. The powdered mix of claim 1 comprising from about 10 to 20% by weight of said flowability enhancer.

6. The powdered mix of claim 1 wherein said phase change material is selected from the group consisting of water, hydrocarbons, alcohols, glycerine, ethylene glycol, polyethylene glycol, clathrates, semi-clathrates, gas clathrates, hydrated salts, and combinations thereof.

7. The powdered mix of claim 1 wherein said phase change material comprises water.

8. The powdered mix of claim 1 wherein said precipitated silica particles have a surface area of from about 140 to about 190 m²/g.

9. The powdered mix of claim 1 wherein said precipitated silica particles have an average particle size of about 250 microns.

10. A therapy pack comprising:
A sealed outer flexible polymeric film containing a powdered mix of a phase change material, a precipitated silica, and a flowability enhancer comprising a carbonate mineral.

11. The therapy pack of claim 10 wherein said outer polymeric film is selected from the group consisting of polyesters, butadiene acrylonitrile copolymers, vinyl polymers, polyethylene, and ethylene-vinyl acetate copolymers.
